(19) United States
(12) Patent Application Publication
(10) Pub. No.: US 2004/0259167 A1
(43) Pub. Date: Dec. 23, 2004

(54) Isolated osteocalcin fragments

(76) Inventors: Jukka Hellman, Turku (FI); Sanna-Maria Kakonen, Turku (FI); Matti Karp, Littoinen (FI); Timo Lovgren, Turku (FI); H. Kalervo Vaananen, Turku (FI); Kim Pettersson, Turku (FI)

Correspondence Address: ROTHWELL, FIGG, ERNST & MANBECK, P.C., 1425 K STREET, N.W., SUITE 800, WASHINGTON, DC, 20005, US

(21) Appl. No.: 10/895,937

(22) Filed: Jul. 22, 2004

Related U.S. Application Data
(62) Division of application No. 09/462,931, filed on Jan. 18, 2000, now abandoned.
Application No. 09/462,931 is a 371 of international application No. PCT/FI98/00550, filed on Jun. 24, 1998.

(30) Foreign Application Priority Data
Aug. 15, 1997 (FI) 973371

(51) Int. Cl. G01N 33/53

(52) U.S. Cl. 435/7.1

(57) ABSTRACT

The invention relates to an isolated osteocalcin fragment derived from human urine, said fragment being characterized in that at least one of the glutamic acids in the position 17, 21 and 24 of the amino acid sequence

```
                                        6 7
Tyr-Leu-Tyr-Gln-Trp-Leu-Gly-Ala-Pro-Val-Pro-Tyr-
                                17 21 24
Pro-Asp-Pro-Leu-Glu-Pro-Arg-Arg-Glu-Val-Cys-Glu-
                                    30
Leu-Asn-Pro-Asp-Cys-Asp-Glu-Leu-Ala-Asp-His-Ile-
Gly-Phe-Gln-Glu-Ala-Tyr-Arg-Arg-Phe-Tyr-Gly-Pro-
Val
```

(SEQ ID NO:2) is gamma-carboxylated. The invention further relates to a monoclonal antibody or recombinant antibody fragment capable to bind said fragment, a cell line producing said monoclonal antibody, and an immunoassay for quantitative determination of said fragment. Furthermore, the invention relates to a method for the measurement of the rate of bone turnover (formation and/or resorption) and/or for the investigation of metabolic bone disorders.

### CROSS REFERENCE TO RELATED APPLICATION

The present application is a division of U.S. patent application Ser. No. 09/462,931 filed 18 Jan. 2000, which in turn is a national stage filing under 35 U.S.C. §371 of PCT/FI98/00550 filed on 24 Jun. 1998 which in turn claims priority to Finnish patent application No. 973371 filed on 15 Aug. 1997.

### FIELD OF THE INVENTION

This invention relates to an isolated osteocalcin fragment derived from human urine, a monoclonal antibody or recombinant antibody fragment capable to bind said fragment, a cell line producing said monoclonal antibody, and an immunoassay for quantitative determination of said fragment. Furthermore, the invention concerns a method for the measurement of the rate of bone turnover (formation and/or resorption) and/or for the investigation of metabolic bone disorders.

### INTRODUCTION AND BACKGROUND

The publication and other materials used herein to illuminate the background of the invention, and in particular, cases to provide additional details respecting the practice, are incorporated by reference.

Human osteocalcin (hOC), also designated bone Gla protein (BGP), is the most abundant noncollagenous protein synthesized by bone osteoblast (Poser et. al. J Biol Chem 1980; 255:8685-91). Although most of the synthesized osteocalcin is absorbed to bone hydroxyapatite by γ-carboxylated glutamic acids (Gla), a small part of it leaks into the blood stream where it can be detected (Price et al. J Biol Chem 1981; 256:12760-6). Part of the hOC found in blood is also thought to originate from the resorption process, when the hOC inside the bone tissues is released during bone degradation (Gundberg and Weinstein J Clin Invest 1986; 77:1762-7). Levels of circulating hOC have been widely used in the clinical investigations as a marker of bone formation (Power and Fottrell Crit Rev Clin Lab Sci 1991; 28:287-335) and serum hOC levels have been shown to correlate with bone mineral density measurements (Yasamura et al. J Clin Endocrinol Metab 1987; 64:681-5).

The discordant results obtained from different hOC assays have hindered widespread usage of hOC in clinical applications (Masters et al. Clin Chem 1994; 40:358-63, Deftos et al. Clin Chem 1992; 38:2318-21, Delmas et al. J Bone Miner Res 1990; 5:5-11 and Diego et al. 1994; 40:2071-7). This phenomenon could partly be explained due to different assay formats i.e. sandwich vs. competitive assays or due to different detection techniques. Presently no calibration standard is available. However, even if the same standard preparation is used, hOC levels measured in different laboratories cannot be directly compared (Delmas et al. J Bone Miner Res 1990). The diversity of hOC molecule itself in circulation has an evident contribution to its immunoreactivity in various assays. The vitamin K dependent γ-carboxylation degree of the glutamic acid residue varies (Poser et. al. J Biol Chem 1980; 255:8685-91). Impairment of γ-carboxylation of hOC purified from bone has been indicated by Cairns and Price, J Bone Min Res 1994; 9:1989-97 and confirmed in our studies (Hellman et al. J Bone Miner Res 1996; 11:1165-75). When Ca²⁺ binds to Gla residues an α-helix structure is known to form (Hauschka and Carr, Biochemistry 1982; 21:2538-47 and Atkinson et al. Eur J Biochem 1995; 232:515-21). Upon removal of Ca²⁺ with EDTA this helical conformation is destroyed. The conformation of decarboxylated OC lies somewhere between the random coil and helical form. Thus, in solution the peptide occurs as a flexible structure and a single conformation cannot be defined for it (Atkinson et al. Eur J Biochem 1995; 232:515-21). Peptide bonds between arginine residues 19 and 20 and between residues 43 and 44 are susceptible to tryptic hydrolysis leading to peptides 1-19, 20-43, 45-49, 1-43, and 20-49 which may be the main products of hOC breakdown in the circulation (Farrugia and Melick, Calcif Tissue Int 1986; 39:234-8, Hellman et al. J Bone Miner Res 1996; 11:1165-75 and Garnero et al. J Bone Miner Res 1994; 9:255-4).

Multiple immunoreactive forms of hOC have been discovered in circulation (Garnero et al. J Bone Miner Res 1994; 9:255-4) and also in urine (Taylor et al. J Clin Endocrin Metab. 1990; 70:467-72). The fragments of hOC can be produced either during osteoclastic degradation of bone matrix or as the result of the catabolic breakdown of the circulating protein after synthesis by osteoblasts. There is evidence that the production of some of the fragments found in urine occurs before renal clearance and is not a result of it (Taylor et al. J Clin Endocrin Metab. 1990; 70:467-72). Because of the rapid clearance from the circulation by glomerular filtration, the shOC (serum human OC) could reflect the acute changes in bone metabolism, while some of the uhOC (urine human OC) fragments might serve as an index of long term changes (Price et al. J Biol Chem 1980; 256:12760-6).

The first reported measurement of urine osteocalcin (Taylor et al. J Clin Endocrin Metab. 1990; 70:467-72) is based on competitive RIA utilizing polyclonal guinea pig antihuman OC antibodies for recognizing the immunoreactive OC fragments (Taylor et al. Metabolism 1988; 37:872-7.). The assay is said to be specific for the midmolecule epitope of hOC molecule according to information obtained from crossreactivity tests with tryptic fragments and synthetic peptide. The probable epitope for polyclonal antibody recognition is determined quite widely by the authors. It is concluded that the binding site of antisera is located in the midmolecule of the protein and probably involves amino acid 19 and at least a portion of the N-terminal sequence of the 20-43 tryptic digest fragment prior to amino acid 37. The assay is unable to distinquish decarboxylated hOC from carboxylated hOC in other words it is not dependent on the γ-carboxylation degree of glutamic acids 17, 21 and 24 in hOC. Furthermore the detailed characterization of the fragments detected by the assay is missing. In addition, this assay is not suitable for routine measurement of urine because the desalting of urine samples before measurement is inevitable for the proper function of RIA. Because of the titer of immunoresponse to hOC varied remarkably with the individual animals, batch-to-batch variations in antibody production are likely to occur, which in term reduces the reproducibility of the assay. With this assay, in both serum and urine multiple immunoreactive OC-fragments have been detected. In addition, multiple fragments were found in normal adult urine that were not detected in normal adult serum. However, the observed immunoreactive fragments were not characterized in detail. uhOC is better able to distinquish between children with high bone turnover and normal adults than serum hOC. The correlation between the serum and the urine samples as measured by the uhOC RIA was good indicating that the assay is detecting osteocalcin originating from the formation process (r=0.83, p<0.01). Besides this, even better correlation was obtained between uhOC and serum alkaline phosphatase measurements. (Taylor et al. J Clin Endocrin Metab. 1990; 70:467-72). A disadvantage of using shOC as a bone metabolism marker is the obvious diurnal variation of hOC concentration (Gundberg et al. J Clin Endocrinol Metab 1985; 60:736-9). One solution for the problem might be to determine the hOC values in 24-hour urine pool.

In the menopause the concentration of serum osteocalcin is increased. The level of increase is partly dependent on the differences in the osteocalcin assays employed or the population studied, but is generally about 30-50% above the premenopausal values (Ravn et al. Bone 1996; 19:291-8, Bonde et al. J Clin Endocrinol Metab 1995; 80:864-8, Garnero et al. J Bone Miner Res 1996; 11:337-49, Åkesson et al. J Bone Miner Res 1995; 1823-9). Generally, the concentration of hOC decereases during antiresorptive treatment like hormon replacement therapy (HRT) (Chen et al. J Bone Miner Res 1996; 11:1784-92, Hodsman et al. J Clin Invest 1993; 91:1138-48). Serum hOC measurements are therefore utilized for monitoring the effectiveness of treatment and also in the pharmaceutical studies designing new antiresorptive drugs. High turnover in bone metabolism in children and especially, in puberty increases the hOC concnetrations remarkably (Taylor et al. J Clin Endocrin Metab. 1990; 70:467-72, Jaouhari et al. Clin Chem 1992; 38:1968-74, Gundberg et al. Clin Chim Acta 1983; 128:1-8).

### SUMMARY OF THE INVENTION

This invention relates to an isolated osteocalcin fragment derived from human urine, said fragment being characterized in that at least one of the glutamic acids in the position 17, 21 and 24 of the amino acid sequence

6 7
Tyr-Leu-Tyr-Gln-Trp-Leu-Gly-Ala-Pro-Val-Pro-Tyr-Pro-Asp-Pro-Leu-
17 21 24 30
Glu-Pro-Arg-Arg-Glu-Val-Cys-Glu-Leu-Asn-Pro-Asp-Cys-Asp-Glu-Leu-Ala-
Asp-His-Ile-Gly-Phe-Gln-Glu-Ala-Tyr-Arg-Arg-Phe-Tyr-Gly-Pro-Val

(SEQ ID NO:2) is gamma-carboxylated.

According to another aspect, the invention relates to a monoclonal antibody or recombinant antibody fragment having the capability of binding the human gamma-carboxylated osteocalcin fragment as defined above.

According to a third aspect, the invention concerns a cell line producing said monoclonal antibody.

According to a fourth aspect, the invention relates to an immunoassay for quantitative determination of a gamma-carboxylated osteocalcin fragment defined above, said immunoassay being characterized in that a sample containing said fragment is exposed to a monoclonal antibody or recombinant antibody fragment which binds said gamma-carboxylated osteocalcin fragment.

According to a fifth aspect, the invention relates to a method for the measurement of the rate of bone turnover (formation and/or resorption) and/or for the investigation of metabolic bone disorders in an individual, said method being based on the quantitative determination of an osteocalcin fragment as defined above.

This is the first report of isolation and characterization of osteocalcin fragments in urine. Three two-site assay detecting these hOC fragments in urine utilizing well characterized reagents are described and validated with clinical samples. The described non-competitive immunoassays are the first assays sensitive enough for the detection of urine osteocalcin fragments in routine clinical measurements. Urine hOC discriminates the pubertal subjects from the adult subjects better than the serum hOC. In addition, an outstanding clinical utility for the discrimination of the postmenopausal group from the premenopausal group is observed. Furthermore, hOC concentrations in postmenopausal group receiving antiresorptive treatment are significantly lower when compared to the concentrations in control postmenopausal group. Serum and urine samples from the same individuals do correlate using the same assay but substantial differences do occur. This result indicates that the fragments of hOC in urine might reflect different state of bone metabolism than fragments of hOC in serum.

### BRIEF DESCRIPTION OF THE DRAWINGS

FIGS. 1A and 1B show amino acid sequences and plasmid vectors. FIG. 1A shows the nucleic acid (SEQ ID NO:1) and the amino acid (SEQ ID NO;2) sequences of the synthetic human osteocalcin insert (SEQ ID NO:3). FIG. 1B shows plasmid vector pGEX-3X (Pharmacia). The arrow indicates the SmaI-ligation site of the hOC insert. The pfXa (protease factor Xa) cleavage site is located after the Ile-Glu-Gly-Arg-sequence (residues 1-4 of SEQ ID NO:4).

FIGS. 2A and 2B shows SDS-PAGE and Western blotting analysis, respectively, of various osteocalcin forms. Lane 1. Low molecular weight (kDa) markers, lane 2. Affinity-purified GST (glutathione S-transferase), lane 3. hOC purified from human bone, lane 4. bOC (bovine osteocalcin), lane 5. Affinity-purified GST-rhOC (GST-recombinant human osteocalcin fusion protein), lane 6. Chromatographically purified rhOC (recombinant human osteocalcin) i.e. the cleavage product from incubation of GST-rhOC with pfXa.

FIG. 3 is a schematic representation of the approximate epitopes recognized by the Mabs used in two-site hOC assays. The molecule has been divided into four epitope areas each of which is being recognized by different Mabs. Circled numbers indicate the number of the hOC specific immunoassay. Amino- and carboxyterminal amino acids have been marked as 1 and 49, respectively. The protease-sensitive sites have been indicated as R-R (arginine-arginine), the three Gla-residues are shown as well as the disulphide bridge (C-C).

FIG. 4 shows the determination of immunoreactive uhOC fragments in normal pubertal urine. Urine was subjected to immunoaffinity chromatography and solid phase extraction before HPLC fractionation. The fractions were measured for immunoreactive material with hOC specific assay #7. The squares refer to osteocalcin and the triangles to acetonitrile.

FIGS. 5A to 5E show characteristics of the immunoreactive hOC fragments isolated from normal pubertal urine. FIG. 5A: Mass analysis of the most prominent fragment 44 isolated from urine spanning the amino acid residues 7-30 (Gly-Asp). FIG. 5B: Mass analysis of the urine hOC fragment 46 spanning the amino acid residues 6-30 (Leu-Asp). FIG. 5C: Mass analysis of the urine hOC fragment 43. FIG. 5D: Mass analysis of the urine hOC fragment 47. FIG. 5E: Characteristics of the hOC fragments isolated in urine and characteristics of intact hOC.

FIG. 6 shows the difference between the hOC concentrations in serum and urine between pubertal and adult samples as measured by the hOC IFMAs. hOC concentration in urine and serum samples was clearly higher in pubertal girls than in premenopausal women as measured with the hOC immunoassays.

FIGS. 7A and 7B demonstrate hOC levels in pre-, postmenopausal and postmenopausal with HRT groups of women measured with the IFMAs. The concentrations have been obtained in serum (FIG. 7A) and urine (FIG. 7B) samples of adult females. In the urine samples the differences between menopausal groups were more obvious than in the serum samples even as measured with the same combination of Mabs.

FIGS. 8A to 8D show correlations between different assays measured in urine and serum samples from adult female panel. FIG. 8A: Correlation between uhOC as measured by the assays #4 and #7. FIG. 8B: Correlation between uhOC as measured by the assays #7 and #9. FIG. 8C: Correlation between serum and urine samples as measured by the assay #7. FIG. 8D: Correlation between serum and urine samples as measured by the assay #4.

### DETAILED DESCRIPTION OF THE INVENTION

According to a preferred embodiment, the isolated osteocalcin fragment derived from human urine is a fragment spanning i) from the amino acid in position 7 to the amino acid in position 30, or ii) from the amino acid in position 6 to the amino acid in position 30 of the amino acid sequence

6 7
Tyr-Leu-Tyr-Gln-Trp-Leu-Gly-Ala-Pro-Val-Pro-Tyr-Pro-Asp-Pro-Leu-
17 21 24 30
Glu-Pro-Arg-Arg-Glu-Val-Cys-Glu-Leu-Asn-Pro-Asp-Cys-Asp-Glu-Leu-Ala-
Asp-His-Ile-Gly-Phe-Gln-Glu-Ala-Tyr-Arg-Arg-Phe-Tyr-Gly-Pro-Val

(SEQ ID NO:2) where all three glutamic acids in the positions 17, 21 and 24 of said sequence are gamma-carboxylated.

The preferred monoclonal antibody or recombinant antibody fragment has a specificity to epitopes that have been identified on the gamma-carboxylated fragment of osteocalcin, wherein said fragment spans either

i) from the amino acid in position 7 to the amino acid in position 30, or

ii) from the amino acid in position 6 to the amino acid in position 30

of the amino acid sequence described above, and that all three glutamic acids in the positions 17, 21 and 24 of said sequence are gamma-carboxylated.

The preferred immunoassay according employs a monoclonal antibody or recombinant antibody fragment having the said specificity.

The preferred immunoassay is a non-competitive immunoassay employing at least two different monoclonal antibodies or recombinant antibody fragments.

The non-competitive immunoassay is preferably carried out in either a one-step or a two-step incubation procedure.

Particularly preferable immunoassays are those where the two monoclonal antibodies employed are

i) the antibodies 2H9 and 6F9 that recognize the C-terminal and N-terminal epitopes on the fragment which was determined to be 3005.

ii) the antibodies 6F9 and 1C4 that recognize the N-terminal and the C-terminal epitopes on the measured osteocalcin fragments (6-30 or 7-30), or

iii) the antibodies 6F9 and 3H8 that recognize the N-terminal and the C-terminal epitopes on the measured osteocalcin fragments (6-30 or 7-30).

All the disclosed assays are highly sensitive and are based on widely characterized reagents. Difference in hOC concentration between the premenopausal and the pubertal group was clearly higher in urine samples than in serum samples. All the hOC assays discriminated the menopausal groups effectively using either serum or urine specimens. Due to their ability to detect different hOC forms, these assays should be of interest in monitoring various disease states, particularly of bone metabolism diseases. The assays are thus especially useful in methods for the measurement of the rate of bone turnover (formation and/or resorption) and/or for the investigation of metabolic bone disorders.

### EXPERIMENTAL

1. Production of the Recombinant Osteocalcin Fusion Protein

Materials

Molecular biology reagents and enzymes were obtained from Pharmacia Biotech, Uppsala, Sweden or from New England Biolabs. Expression vector pGEX-3X was obtained from Pharmacia Uppsala, Sweden. Escherichia coli XL1-Blue strain (recA1, endA1, gyrA96, thi1, hsdR17, supE44, relA1, lac, F′ proAB, lacI^{q}ZDM15, Tn10 (tet^{r})) was used for the expression of the GST-rhOC fusion protein. L-broth culture medium contained 10 g/l Bacto® Tryptone (Difco laboratories, Michigan, USA), 5 g/l Bacto® Yeast extract (Difco) and 5 g/l NaCl, pH 7.4. Isopropyl-1-thio-β-D-galactoside, IPTG (Sigma Chemical CO, USA) was used for induction. PBS buffer consisted of 150 mM NaCl, 16 mM Na₂HPO₄, 4 mM NaH₂PO₄, pH 7.3. PMSF and reduced glutathione were obtained from Sigma and protease factor Xa, pfXa from New England Biolabs. Glutathione Sepharose® 4B column (bed volume 8 ml) was obtained from Pharmacia. The size separation of the proteins was done with SDS-PAGE 25% gradient Phastgel and using Low molecular weight markers for standardization (Pharmacia). Bovine osteocalcin (bOC) was obtained from Biodesign International, Kennebunkport, Me. A commercial anti-bOC Mab BD (Biodesign) was used as a primary antibody and a horseradish peroxidase linked anti-mouse immunoglobulin raised in sheep was used as a second antibody (Amersham, Buckinghamshire, England). ECL Western blotting reagents (Amersham) were used for visualization according to manufacturer's suggestions.

Equipment

Human osteocalcin oligomers and the oligomer primers designed for sequencing were synthesized on an Applied Biosystems (Foster City, Calif.) oligonucleotide synthesizer. Nucleid acid sequencing was done with T7 Sequencing Kit and Macrophor equipment from Pharmacia. The electroporation was done with Gene pulser (Bio-Rad, Richmond, Calif.). Sodium dodecyl sulfate-polyacrylamide gel electrophoresis (SDS-PAGE) was run with PhastSystem (Pharmacia). Matrix assisted laser desorption (MALDI-MS) mass spectrometer (LASERMAT^{R}, Finnigan MAT Ltd., U.K.) was employed to obtain the mass of the peptides. A protein sequencer (Applied Biosystems model 477A) equipped with an on-line Applied Biosystems model 120A PTH amino acid analyzer was employed for the NH₂-terminal amino acid analyses. Reverse phase chromatography was done using a C8 RP-300 column (4.6×100 mm) (Applied Biosystems). The proteins were electroblotted onto Hybond™-C extra nitrocellulose membranes (Amersham) using PhastTransfer™ Semi-dry Transfer Kit (Pharmacia) according to PhastSystem™ manual.

Plasmid Construction

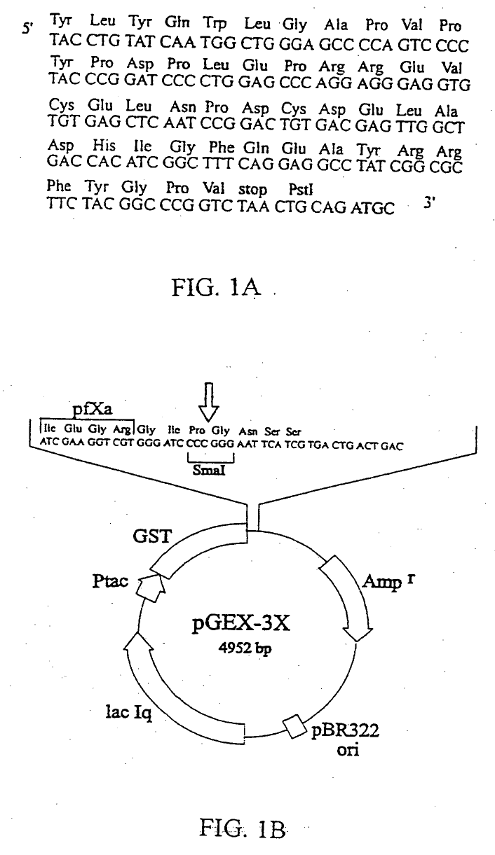

Synthetic human osteocalcin oligomers (FIG. 1A), each containing 88 nucleic acids (8 of them being complementary to each other) were hybridized at RT after phosphorylation of the ends. The single-stranded ends were filled by 1 U of Klenow polymerase and 100 μM of each deoxynucleotide (30 minutes incubation at 37° C.) to create a 160 base pair human osteocalcin insert, which contains a stop codon and a PstI restriction enzyme site at the 3′ end of the gene (FIG. 1A). The blunt-ended insert was ligated in SmaI-digested, dephosphorylated prokaryotic expression vector, pGEX-3X (FIG. 1B). The recombinant plasmid was transformed into E. coli and the resulting vector was confirmed by restriction enzyme digestion and nucleic acid sequencing. All molecular biology protocols were according to Sambrook et al. Molecular Cloning, a Laboratory Manual, Second Edition, 1989, Cold Spring Harbor Laboratory Press, Cold Spring Harbor, N.Y.

Expression of the GST-rhOC Fusion Protein

Transformed E. coli cells were grown at 37° C. in 250 ml of L-broth containing 100 μg/ml ampicillin to an A₆₀₀=0.5 and then IPTG was added to a final concentration of 0.5 mM. Induced cells were grown for an additional 2 h and were collected by centrifugation, disrupted by sonication on ice in PBS buffer containing 2 mM PMSF, Sigma and 1 mM EDTA as protease inhibitors. After sonication, Triton-X-100 (1% v/v) was added. The sonicate was clarified by centrifugation (10,000 g, 20 min, 4° C.) and filtered through a 0.45 μm filter before applying it to an equilibrated Glutathione Sepharose® 4B column according to manufacturer's suggestions.

Purification of the Recombinant Human Osteocalcin

After concentration, the eluate containing the GST-rhOC fusion protein was incubated with pfXa in 20 mM Tris-HCl, 100 mM NaCl, 2 mM CaCl₂, pH 8.0, using a protease to substrate ratio of 0.5% (w/w) for 30 min at RT to release the rhOC portion (Nagai and Thøgersen Nature 1984; 309:810-2). Then rhOC was separated from the mixture reverse phase chromatography using a C8 RP-300 column. The optimization of the rhOC purification has been described in detail by Käkönen et al. (1996).

Verification of the Protein Products

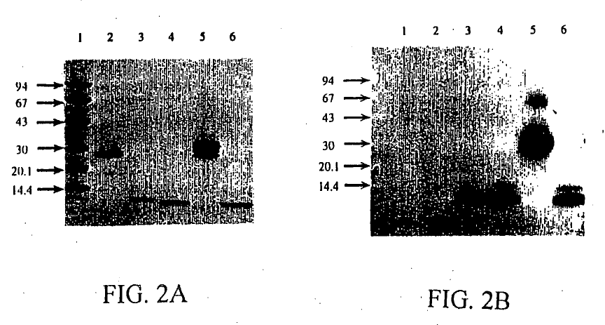

The expression vector contained the entire sequence of the hOC gene fused in frame to the 3′ end of the Schistosoma japonicum glutathione S-transferase gene (Smith and Johnson Gene 1988; 67:31-40) as verified by nucleic acid sequencing. The resulting fusion protein contains three additional amino acids between the protease factor Xa cleavage site and the first amino terminal tyrosine of the hOC gene (FIG. 1B The molecular mass of the reverse phase HPLC purified rhOC was 6068.1 as determined by mass spectrometry. This value is consistent with the predicted mass calculated from the amino acid sequence (6064.8, rhOC has a extension of three aminoacid residues (Gly-Ile-Pro) because of the of pfXa cleavage site in pGEX-3X plasmid (FIGS. 1A and 1B.). Purified GST-rhOC and rhOC were compared with hOC purified from bone and with bOC using SDS-PAGE and Western blotting analysis. The size of purified GST-rhOC (32 kDa) obtained by SDS-PAGE is in accordance with that expected from a fusion protein containing GST (26 kDa, Smith and Johnson Gene 1988;. 67:31-40) and rhOC (6068.1 Da). Purified rhOC migrates similarly both to hOC purified from human femurs and bOC on SDS-PAGE (FIG. 2A, lanes 3, 4 and 6, respectively). In Western blotting experiment, Mab BD binds to hOC and bOC (FIG. 2B, lanes 3 and 4, respectively) and also to the both recombinant forms, GST-rhOC and rhOC (FIG. 2B, lanes 5 and 6, respectively). A 64 kDa band is also observed from the fusion protein sample (FIG. 2B, lane 5) which probably indicates a dimeric form of GST-rhOC. Mab BD does not bind to GST or to any of the molecular weight markers (FIG. 2B, lanes 1 and 2, respectively).

2. Production of the Osteocalcin Monoclonal Antibodies

Materials and Equipment

GST-rhOC used as an immunogen was produced as described (Käkönen et al. Prot Exp Purif 1996; 3:137-44). Bovine osteocalcin (bOC) was obtained from Biodesign International, Kennebunkport, Me. Freund's complete adjuvant (Fca) and Freund's incomplete adjuvant (Fia) were obtained from Sigma Immuno Chemicals, St Louis, Mo. The Optimem-1 with glutamax-1, Dulbecco's Modified Eagle Medium (DMEM, 10×liquid), L-glutamine, sodiumpyruvate (tissue culture tested), sodiumbicarbonate (tissue culture grade) and penicillin-streptomycin (P/S) solution were purchased from Gibco BRL, Life Technologies, Grand Island, N.Y., Hepes from Boehringer Mannheim, Germany and the fetal bovine serum from Hyclone, Logan, Utah and were used as components in culture mediums. Reagents and equipment used for screening the hOC specific hybroma cell lines were obtained from Wallac Oy, Turku, Finland except the Europium-labeled hOC was prepared according to Hellman et al. (J Bone Miner Res 1996; 11:1165-75).

Pristane (2,6,10,14-tetramethyl pentadecan) used for the production of Mabs as ascitic fluid was obtained from Aldrich-Chemie, Steinhiem, Germany). Tecnomouse hollow-fiber bioreactor used for the large scale production of Mabs was obtained from Integra Biosciences AG, Wallisellen, Switzerland and Protein A agarose Affigel® for the purification of the Mabs was from Bio-Rad, Richmond, Calif.

Immunization of the Mice and Screening of the hOC Specific Mabs

The employing of recombinant OC fusion protein as an immunogen has been described earlier (Matikainen et al In “Animal cell technology: Developments towards the 21st century” 1995; pp. 475-9). Ten months old male Balb/c mouse was intraperitoneally immunized with 413 μg GST-rhOC antigen (corresponding to 75 μg of rhOC portion) mixed with Fca. The mouse was boostered 15 weeks later with 358 of the same antigen (60 μg of rhOC) mixed in Fia. The final booster dose, 110 μg antigen (20 μg of rhOC) in PBS was given i.p. after 8 weeks.

Bovine osteocalcin was coupled to keyhole limpet hemocyanin (KLH) as described (Young et al. Prostaglandines 1982; 23:603-13). Two three-month-old Balb/c male mice were i.p. immunized with 50 μg of bOC-KLH antigen mixed with Fca. The mice were boostered with the same amount of antigen in Fia. The final booster dose, 10 μg bOC-KLH in PBS, was given intravenously.

Three days after the final boostered the splenocytes were fused to mouse myeloma cells SP 2/0 as described in more detail earlier (Lilja et al. Clin Chem 1991; 37:1618-25). The hybridomas were grown in Optimem-1 with glutamax-1 containing 20% of fetal bovine serum. The hOC specific Mabs were screened with immunofluorometric assay (IFMA) using rabbit antimouse Ig microtitration wells and hOC labeled with Europium as described earlier (Matikainen 1995).

Large Scale Production and Purification of the Mabs

Before large scale production of Mabs the positive hybridomas were cloned at least twice by the limiting dilution method. Optimem-1 with glutamax-1 supplemented with 20% of fetal bovine serum was used as culture medium. Cell lines 2H9F11F8 (2H9) and 6F9G4E10 (6F9) obtained from the GST-rhOC immunization and 3G8E1F11 (3G8), 1C4B1D7E7 (1C4) and 3H8H2D2A12F12 (3H8) obtained from bOC immunizations were selected for further characterization. The Mabs were produced as ascites fluid in Balb/c mice primed with pristane and in Tecnomouse hollow-fiber bioreactor. DMEM (1×solution) supplemented with L-glutamine, Hepes, sodiumpyruvate, sodiumbicarbonate and P/S was used as a culture medium in intracapillary circulation. Optimem-1 with glutamax-1 supplemented with 2.5% fetal bovine serum was used as a harvesting medium in extracapillary space. Produced Mabs were purified by Protein A agarose chromatography using Affigel® purification kit according to manufacturer's suggestions.

3. Epitope Mapping, Characterization of the Mabs and Two-Site Assays

Materials

In the subclass determination of the Mabs, the streptavidin-coated microtiter plates were obtained from Wallac Oy and biotinylated rat antimouse Ig subclass specific Mabs from Serotec, Oxford, England. For epitope mapping the synthetic osteocalcin peptide 7-19 containing Glu at residue 17 was purchased from Bachem, Switzerland and bovine osteocalcin (bOC) was obtained from Biodesign International, Kennebunkport, Me. Osteocalcin from human femurs was purified by modifying a previously described method (Gundberg et al. Meth Enzymol 1984; 107:516-544) as explained in detail by Hellman et al. (1996). Also the carboxypeptidase Y digestion, trypsin digestion, alkylation and decarboxylation of hOC has been described earlier (Hellman et al. 1996). The detailed characterization of the hOC and its modifications is described by Hellman et al. (1996). The recombinant forms of osteocalcin were produced as explained in section 1. The production and purification of a truncated form of rhOC (del. rhOC) (lacking 10 COOH-terminal amino acid residues) from a gene carrying a stop mutation in the structural region was performed under similar conditions. Both rhOC and del rhOC have an extension of three amino acid residues in their NH₂ termini; in addition, they lack the γ-carboxylation characteristic of hOC isolated from bone.

Reagents for biotinylation, biotin isothiocyanate (BITC) and labeling, europium(III) chelate of 4-[2(-4-isothiocyanatophenyl)ethynyl]-2,6-bis {[N,N-bis(carboxymethyl)amino]-methyl}pyridine (Takalo et al. Helv Chim Acta 1993; 76:877-83) were from Wallac Oy. Streptavidin coated microtiter plates, Delfia® Buffer, Delfia® Wash Solution, Delfia® Enchancement Solution and Delfia® Research Fluorometer, Model 1234 used in IFMA measurements were obtained from Wallac Oy. Reagents and equipment used in the immunoassays were similar throughout the study.

Characterization of the Mabs

Labeling of the peptides for subclass determination and epitope mapping was done according to Hellman et al. 1996. The Mabs 6F9, 3G8, 1C4 and 3H8 belonged to subclass IgG1 and the Mab 2H9 to subclass IgG2a as determined according to Matikainen et al., 1995.

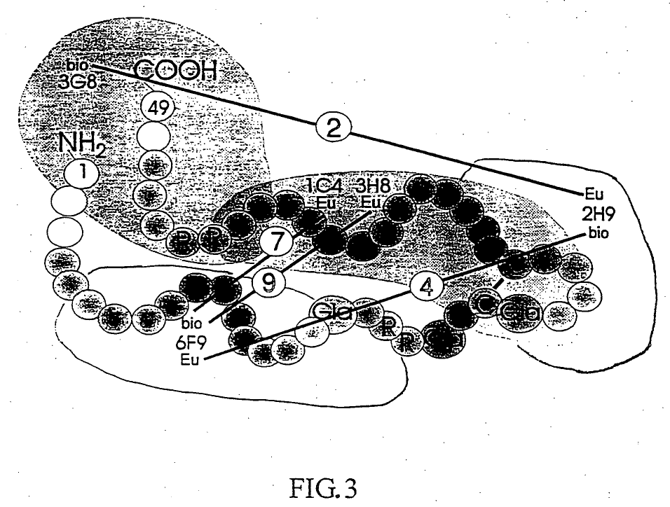

Mabs were characterized for their binding to Eu-labeled intact hOC, bOC and tryptic or synthetic peptides as described (Hellman et al. 1996). The antibodies 1C4 and 3H8 obtained by immunization with bOC conjugated to KLH recognized the tryptic 20-43 fragment. For Mab 3G8, also obtained from bOC immunizations, no specific binding site could be located using labeled peptides. Also, labeling of intact bOC and hOC abolished their immunoreactivity with 3G8, suggesting either that intact Tyr (1) is needed for efficient binding of that the Eu-chelate causes steric hindrance. Unlabeled intact hOC or bOC were, however, easily recognized by this antibody in a two-site format with Mab 2H9. Mab 6F9 recognized the tryptic 1-19 and synthetic 7-19 peptides. Mab 2H9 recognized the tryptic 20-43 peptide. Summary of the Mabs in Table 1. According to information obtained an epitope map was created (FIG. 3).

TABLE 1
Characterization of Mabs against

| Hybridoma | | Immunoglobulin | Eu-labeled OC forms recognized^{b} | | | | | |
|---|---|---|---|---|---|---|---|---|
| clone | Immunogen | class H-chain^{a} | Eu-hOC | Eu-hOC 1-19 | Eu-hOC 7-19 | Eu-hOC 15-31 | Eu-hOC 20-43 | Eu-bOC |
| 2H9 | rGST-hOC | IgG2a | + | − | − | + | + | + |
| 3G8 | bOC-KLH | IgG1 | +^{c} | − | − | − | − | +^{c} |
| 3H8 | bOC-KLH | IgG1 | + | − | − | + | + | + |
| 1C4 | bOC-KLH | IgG1 | + | − | − | + | + | + |
| 6F9 | rGST-hOC | IgG1 | + | + | + | + | − | − |

^{a}Allκ light chains.
^{b}Measured according to Hellman et al. 1996 c Mab 3G8 recognizes unlabeled hOC and bOC when tested with
^{c}Mab 3G8 recognizes unlabeled hOC and bOC when tested with two-site

Biotinylation and Eu Labeling of the Mabs

In order to create two-site combination assays the Mabs 3G8, 2H9 and 6F9 were biotinylated with BITC and the Mabs 2H9, 6F9, 1C4 and 3H8 were labeled with europium(III) chelate in reaction conditions previously described (Hellman et al, 1996). The two-site immunoassay utilized time-resolved fluorometry using lanthanide chelate labels, like europium, as a detection system (Soini and Lövgren CRC Crit Rev Anal Chem. 1987; 18:105-53).

Characterization of the Two-Site Assays

Four of the two-site combinations were validated in more detail by determination of the crossreactivities to the alkylated hOC, decarboxylated hOC, carboxypeptidase Y-digested hOC, recombinant hOC and truncated recombinant hOC. According to information obtained by crossreactivity determination and the epitope mapping the assay # 2 (bio-3G8/Eu-2H9) was considered specific for the full length, intact hOC molecule. The assays # 4 (bio-2H9/Eu-6F9), #7 (bio-6F9/Eu-1C4) and #9 (bio-6F9/Eu-3H8) were able to detect the full length hOC and also the large NH₂-terminal fragment. Assay #4 measured both γ-carboxylated and fully decarboxylated form of hOC. The #9 and #7 assays distinctively preferred the carboxylated form of hOC. Determination of the affinity constants of labeled Mabs were done according to Hellman et al. 1996 using Scatchard analysis (Scatchard, Ann NY Acad Sci 1949; 51:660-72). Characteristics of the assays are summarized in Table 2.

TABLE 2
Characteristics of the two-site IFMAs of

| | | | Kₐ (×10° L/mol)^{a} | | |
|---|---|---|---|---|---|
| Combination | Capture | Tracer | of tracer Mab in | | |
| number | Mab | Mab | AB | +5 mM EDTA | +25 mM Ca |
| 2 | 3G8 | 2H9 | 3.30 | 3.40 | 4.20 |
| 4 | 2H9 | 6F9 | 0.7 | 0.83 | 0.42 |
| 7 | 6F9 | 1C4 | 0.13 | 0.11 | 0.07 |
| 9 | 6F9 | 3H8 | 0.68 | 0.64 | 0.2 |

| Combination | Capture | Tracer | Crossreactivities in percent (w^{b}/w) | | | | | |
|---|---|---|---|---|---|---|---|---|
| number | Mab | Mab | hOC | dec. hOC | rhOC | CPY hOC | del. rhOC | alkyl. hOC |
| 2 | 3G8 | 2H9 | 100 | 64 | 69 | 9 | 1 | 76 |
| 4 | 2H9 | 6F9 | 100 | 46 | 44 | 150 | 65 | 75 |
| 7 | 6F9 | 1C4 | 100 | 19 | 6 | 169 | 18 | 6 |
| 9 | 6F9 | 3H8 | 100 | 8 | 7 | 156 | 6 | 3 |

^{a}Affinities determined according to Scatchard as described by
^{b}Measured as described by Hellman et

In addition to two-site non-competitive assays, the hOC specific antibodies could be utilized competitive assays as a capture antibody. In competitive assay hOC fragments in urine compete with the Eu-labeled hOC for binding to the limited number of capture Mabs. With Mabs 2H9, 1C4 and 3H8 also Eu-labeled bOC could be used due to crossreactivities explained in Table 1.

4. Optimized Assay Procedures

Materials and Equipment

The employed Mabs have been characterized in the previous section. In addition to Mabs produced in hybridoma cell culture, also recombinant antibody fragments could be used in the assay concept. hOC and CPYhOC (Carboxy-peptidase Y digested hOC) used for the standardization of the assays were produced as explained in section 3. DTPA (diethylenetriaminepentaaceticacid)-treated BSA in TSA-buffer (50 mM Tris-HCl, 150 mM NaCl, 15 mM NaN₃, pH 7.75) used as a diluent in standardization was obtained from Wallac Oy. Materials and equipment used in the OC IFMAs have been listed in the section 3.

All-in-One Assays Optimized for Serum Samples

10 μl of samples and standards were pipetted streptavidin coated microtiter plates. The calibration curve was prepared using purified hOC in 7.5% (w/v) DTPA-treated BSA in TSA buffer as a standard covering the range from 0.5 to 80 ng/ml. Then a mixture of biotinylated and Eu labeled Mabs in 50 μl of Delfia® Buffer was added to the wells. The amount of capture or tracer Mab was 200 ng/well except 100 ng/well of tracer Mabs were used in assays #2 and #9. 5 mM EDTA was added into Delfia® Buffer in assays #4, #7 and #9. The plates were shaken for 2 h at RT followed by washing six times with Delfia® Wash Solution. To detect the Eu fluorescence, 200 μl of Delfia® Enhancement solution per well was added. Prior to the measurement with the Delfia® Research Fluorometer the plates were shaken 30 min at RT.

The lower limit of the detection of the different assays were determined based on two standard deviations of the background signal produced by the standard diluent and was under 0.1 μg/L for each assay. The developed IFMAs showed a linear response of over four orders of magnitude and were highly reproducible.

Osteocalcin Assays Optimized for Urine Samples

The calibration curve for assay #4 was prepared using purified hOC purified hOC in 7.5% (w/v) DTPA-treated BSA in TSA buffer as a standard covering the range from 0.05 to 16 ng/ml. Carboxy-peptidase Y digested hOC (CPY hOC) in the same diluent covering the range from 0.05 to 16 ng/ml was used for standardization for the assays #7 and #9.

First, 400 ng of biotinylated capture Mab in 50 μl of Delfia® Buffer was pipetted to the streptavidin well. After 30 minutes shaking at room temperature the excess capture Mab was removed by two washings using Delfia® Wash Solution. Before the adding labeled tracer Mab in 50 μl Delfia® Buffer, the standard or sample was pipetted in 10 μl volume. 100 ng of labeled tracer was used except in assay #7 where 200 ng/well of Eu-1C4 was used. After two hours shaking at room temperature, the wells were washed six times and 200 μl of Delfia® Enhancement solution per well was added. Prior to the fluorescence measurement, the plate was shaken 30 minutes at room temperature. The assays were highly linear and reproducible. The lowest detection limits were under 0.1 μg/L.

5. Isolation and Characterization of Urine Osteocalcin Fragments

Materials and Equipment

Carboxy-peptidase Y digested hOC (CPY hOC) used for standardization of the urine IFMAs was produced as explained in section 3. Immunoaffinity chromatography coupled with purified 6F9Mab (section 2) using Affi-Gel Hz Immunoaffinity kit from Bio-Rad, a C-18 solid phase extraction cartridge (Millipore) and a C-4 reverse phase HPLC column (Vydac, Hesperia, Calif., U.S.A.) were used for the isolation of uhOC fragments. Matrix assisted laser desorption MALDI-TOF mass spectrometer (LASERMAT^{R}, Thermo Bioanalysis Ltd., U.K.) was employed for mass determinations and a protein sequencer (Applied Biosystems model 477A) equipped with an on-line Applied Biosystems model 120A PTH amino acid analyzer was used for the NH₂-terminal amino acid sequence analyses.

Sample Collection and uhOC IFMA

Urine pool was collected in the morning from one healthy male volunteer aged 13 years and stored at +4° C. Within three hours the pool was aliquoted and frozen at −70° C. Later it was stored at −20° C. Urine pool was thawed, centrifuged and filtrated before subjected to any isolation steps.

Immunoreactive uhOC in both urine pool and different steps of isolation process was measured with a two-site immunoassay recognizing, not only the intact hOC, but also the N-terminal mid-fragment of hOC (amino acid residues 1-43) (FIG. 3). The assay used monoclonal antibodies (Mabs) 6F9 and 1C4 as a capture and tracer Mab, respectively (combination #7). The calibration curve was prepared using carboxy-peptidase Y digested hOC (CPY hOC) as a standard. The amount of immunoreactive uhOC in puberty urine pool was 100 ng/ml. Also the assays #9 (Mab combination 6F9/3H8) and #4 (Mab combination 2H9/6F9) were utilized to determine the immunoreactive fragments as described in section 4.

Isolation of Osteocalcin Fragments

First, immunoreactive osteocalcin fragments from puberty urine pool were adsorbed by immunoaffinity chromatography. The gel matrix was covalently coupled with hOC Mab 6F9 recognizing an N-terminal epitope (FIG. 3). After adsorption the bound fragments were eluted by 0.1 M acetic acid and adsorbed onto a C-18 solid phase extraction cartridge, again eluted with 80% acetonitrile (AcN) and evaporated. In the immunoaffinity chromatography, over 90% of the immunoreactive uhOC was adsorbed onto the 6F9 coupled gel. In the elution step (including adsorption steps and washings of the gel) 10% of the adsorbed uhOC was eluted. To improve the yield more urine was applied onto the affinity column and then the eluent was recycled 5 hours through the affinity column attached to two C-18 solid phase extraction cartridges all connected to each others in series. After evaporation the C-18 solid phase eluent contained at least 2 mg/ml of immunoreactive uhOC as measured by the urine hOC assay utilizing Mab combination #7.

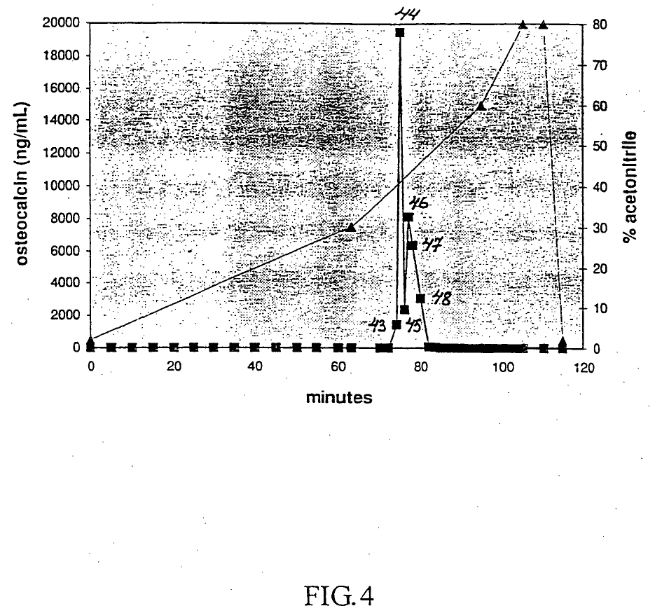

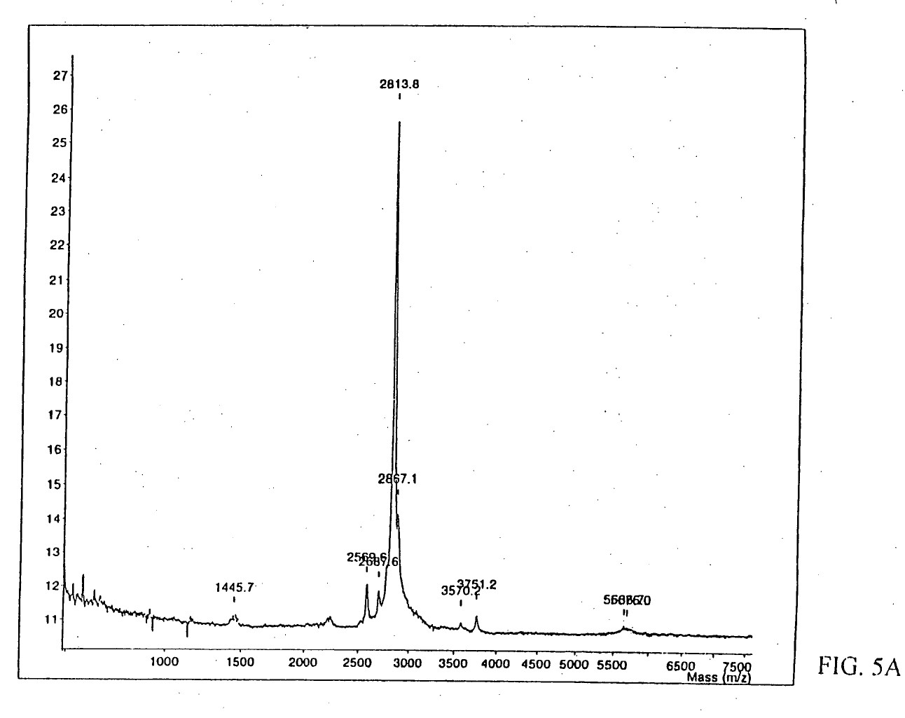

Next, the sample containing the immunoreactive fragments was applied onto a Vydac C-4 (2.1 mm×150 mm) reverse phase HPLC column, eluted with an acetonitrile gradient (FIG. 4) and the peak fractions detected at 276 nm were collected manually. The sample contained multiple immunoreactive fragments of OC, which eluted between 70 min (35% AcN), and 82 min (48% AcN) (FIG. 4). Fractions containing 1.4-19.5 μg/ml of immunoreactive uhOC were subjected to further analysis.

Characterization of Osteocalcin Fragments

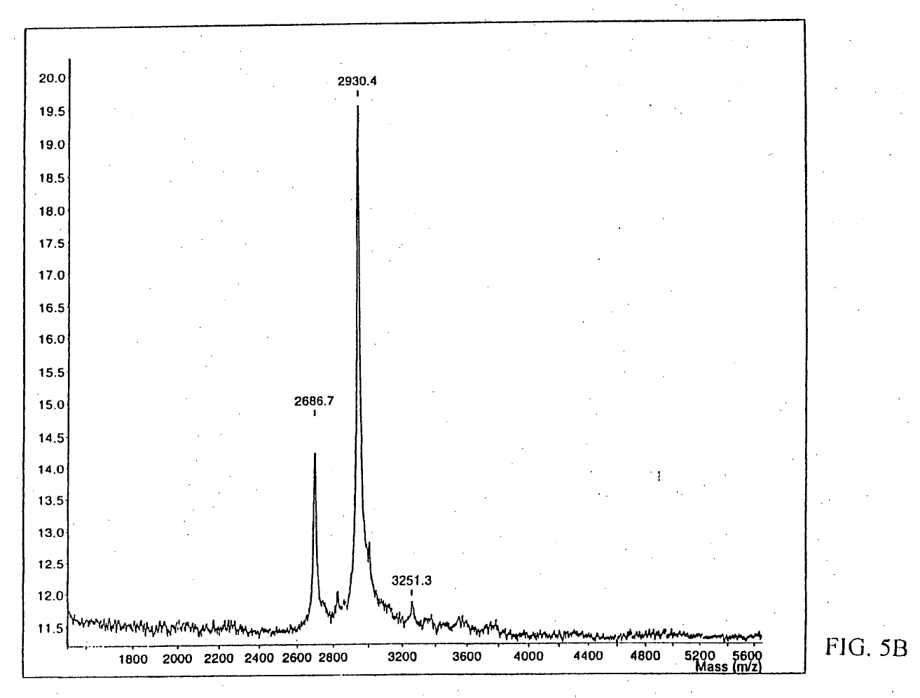

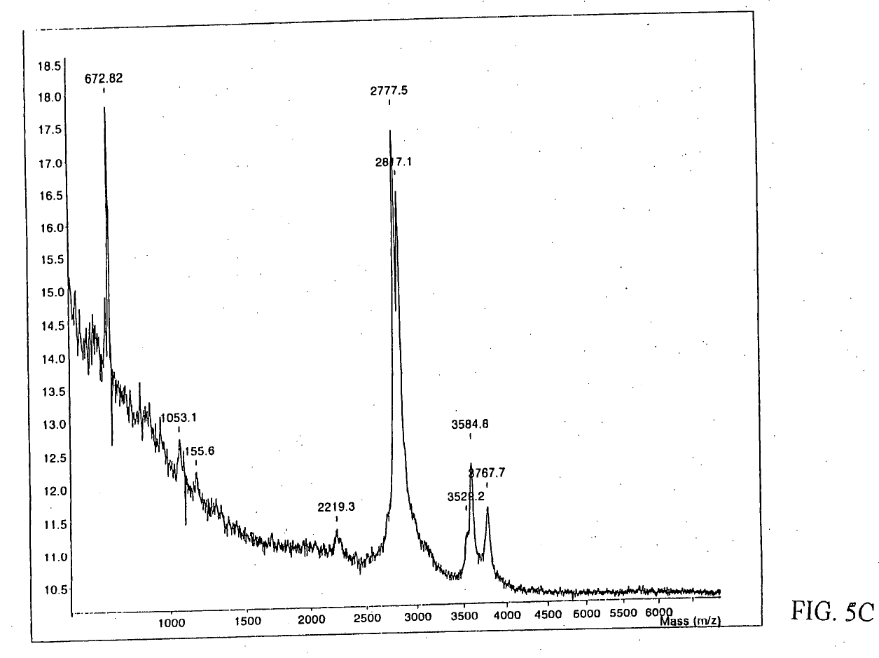

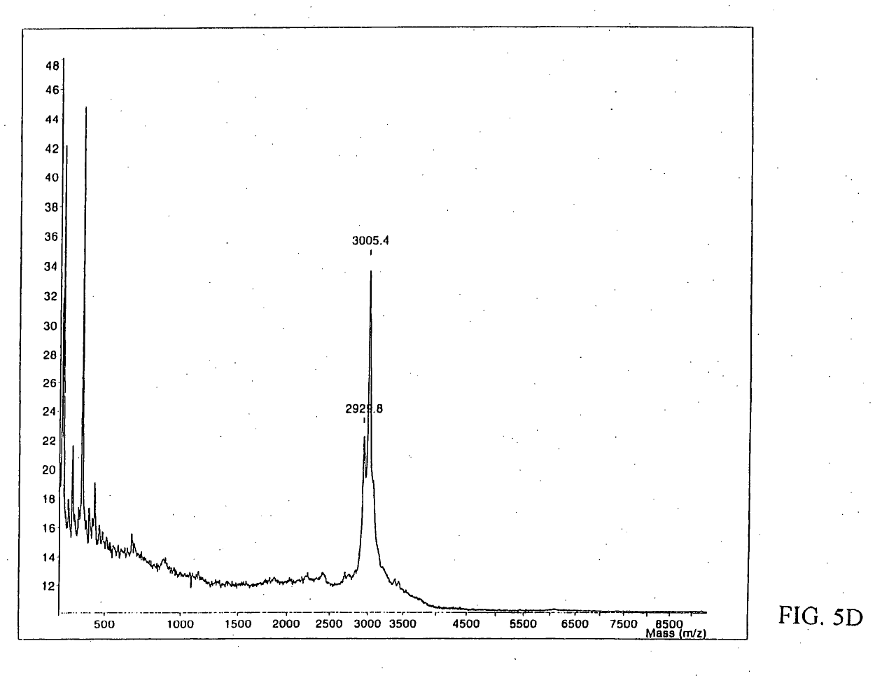

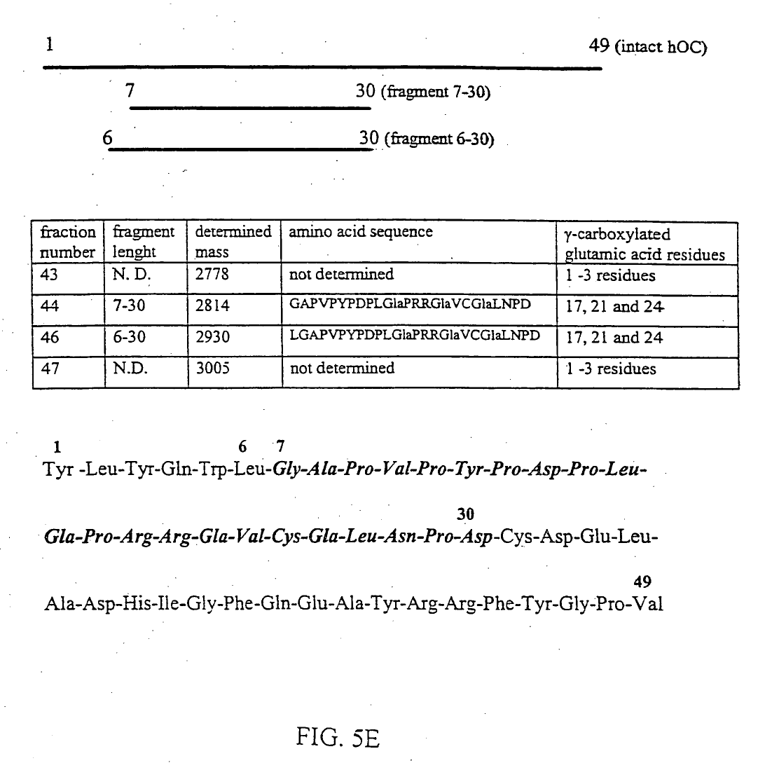

HPLC fractions were analyzed by the two-site immunoassays #7, #9 and #4 as before and fractions containing immunoreactive uhOC fragments were subjected to MALDI-TOF mass spectrometry and N-terminal amino acid sequencing. The molecular masses of the prominent ions in mass spectrometry were 2778, 2814, 2930 and 3005. Fractions 44 (M=2814) and 46 (M=2930) contained enough material for the N-terminal sequence analysis. The sequence obtained from fraction 44 matches with hOC starting from residue Gly(7). Taking into account the experimental mass 2814, the fragment spans residues 7-30, with γ-carboxylated residues at positions 17, 21 and 24 giving a calculated mass of 2812 (FIG. 5A). γ-carboxylation of the Glu residues is further supported by the fact that γ-carboxylated Glu residues are known not to give signal using the sequencing technique in question (Cairns et al. (1991) Anal Biochem. 199, 93-97). Fraction 46 was subjected to trypsin digestion to demonstrate that the fragment can be cleaved as expected (after Arg residue). In addition, the determined N-terminal sequence matches with hOC starting from residue Leu(6). The determined mass of the N-terminal part of the fragment cleaved with trypsin was 1566 and in accordance with the expected mass mass (1565 with γ-carboxylated Glu 17). According to the determined mass of fraction 46 before trypsin cleavage (2930, FIG. 5B) the fragment spans residues 6-30 of hOC with three γ-carboxylated residues as above (calculated mass 2925). The ion species with masses 2778 (FIG. 5C) and 3005 (FIG. 5D) represent close structural variants of the same hOC region, based on immunoreactivity, chromatographic behaviour and molecular mass. Such structural variability can be caused by partial lack of γ-carboxylation or additional 1-2 residues, and/or combinations of both. The characteristics of the fragments have been described in FIG. 5E. In addtion to immunoassay #7, also assay #9 recognize effectively these forms of urine osteocalcin. Epitope of #4 differs slightly from epitope recognized by the combination #7 and #9 because the combination could not recognize the 7-30 fragment but could detect the fragment which mass was 3005. Urine may contain shorter hOC fragments which remain to be characterized.

6. Determination of Osteocalcin Concentrations in Serum and Urine Panels

Materials and Equipment

FSH concentration of serum samples was measured by Delfia® hFSH assay (Wallac, Turku, Finland). The creatinine concentration in urine samples was measured using AU OLYMPUS 510 equipment according to manufacturer's protocols. The protocols of the shOC and uhOC assays have been described in section 5.

Subjects and Sample Collection

Clinical evaluation of the assays was performed with serum and urine samples collected between 8 and 11 o'clock in the morning from 58 pre-, 9 peri- and 20 postmenopausal women, 12 postmenopausal women with hormone replacement therapy (HRT) and 16 pubertal girls. In addition to female panel, also a male panel was collected consisting of 46 adult men and 19 pubertal boys. The serum samples were allowed to clot for 30 min. at room temperature before centrifuging and then immediately aliquoted and stored at −70° C. Collected urine samples were first frozen at −70° C. and then stored at −20° C. The women were divided into pre-, peri- and postmenopausal groups according to menstrual status and FSH concentration in serum. The postmenopausal group was further classified into subjects with or without hormone replacement therapy (HRT).

hOC Concentrations in Serum and Urine Samples

Serum samples were measured with intact hOC assay specific for full-length hOC (#2), with total hOC assay recognizing not only the intact hOC, but also the N-terminal midfragment of hOC (#4) and with two assays dependent on the degree of γ-carboxylation of the glutamic acids in hOC (#7 and #9). The urine samples were measured with two γ-carboxylation dependent assay (#7 and #9) and also with assay #4. The urine osteocalcin values used for analysis have been corrected for creatinine.

shOC and uhOC in Pubertal Subjects Compared to Adult Subjects

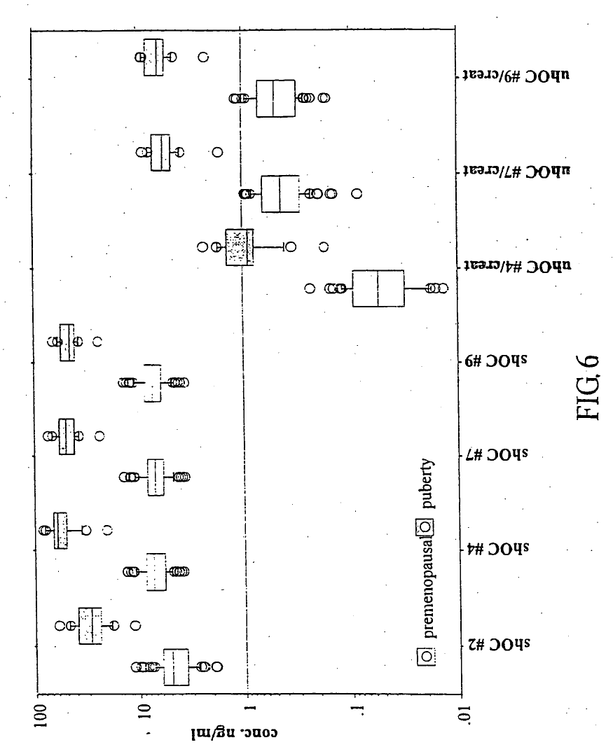

In women the hOC values observed in serum samples were six to eight fold higher in pubertal girls than in adults. In urine, the hOC concentrations were twelve to sixteen fold higher when comparing the pubertal group to the adults. (FIG. 6.). In men the increase was five fold in serum and eight to eleven fold in urine. Although, the hOC concentration level observed by the assay # 4 is approximately five fold lower than the level observed by assays # 7 or # 9, the concentrations differ significantly between examined groups. All increases were highly significant (p<0.001). The accurate values have been summarized in the Table 3.

TABLE 3
The difference in hOC concentrations between
pubertal and adult males and females

| | shOC | shOC | shOC | shOC | uhOC | uhOC | uhOC |
|---|---|---|---|---|---|---|---|
| group | #2 | #4 | #7 | #9 | #4 | #7 | #9 |
| female | 6.3 | 7.8 | 6.9 | 6.2 | 16.5 | 12.3 | 12.5 |
| male | 4.9 | 4.7 | 4.8 | 4.9 | 10.8 | 8.2 | 8.3 |

shOC and uhOC in Different Menopausal Groups

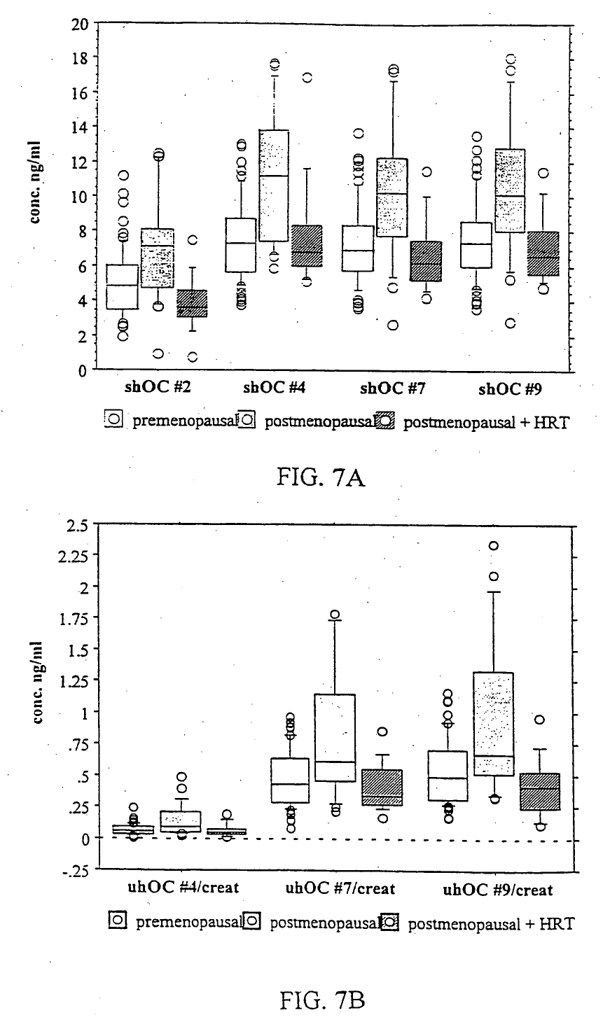

The statistically significant increase in hOC concentrations in serum was observed in menopause (40 to 48%). Interestingly, in urine samples the increase in hOC concentration was as high as 75% and 79% as measured with assays #7 and #9, respectively (p<0.001). The increase of urine hOC between the pre- and postmenopausal groups was even higher when measured with assay #4 (125%, p<0.001). hOC concentrations in postmenopausal subjects on HRT decreased to concentrations indistinguishable from the premenopausal group with every hOC assay in both serum and urine specimens. Statistically significant decreases (30 to 46%) in serum concentrations were seen depending on the assay used. However, the observed decrease in urine samples was over 50%. (FIGS. 7A and 7B.). Although, the hOC concentration level observed by the assay # 4 is approximately five fold lower than the level observed by assays # 7 or # 9, the concentrations differ significantly between examined groups. The discriminatory power of each assay has been summarized in Table 4.

TABLE 4
Percentual differences and statistical significancies between
menopausal groups as measured by hOC IFMAs in serum and urine samples

| | shOC | shOC | shOC | shOC | uhOC | uhOC | uhOC |
|---|---|---|---|---|---|---|---|
| | #2 | #4 | #7 | #9 | #4 | #7 | #9 |
| Increase in | 42 | 48 | 43 | 40 | 125 | 75 | 79 |
| menopause (%) | | | | | | | |
| p-value | 0.0008 | <0.0001 | <0.0001 | <0.0001 | <0.0001 | <0.0001 | <0.0001 |
| Decrease in HRT | 46 | 30 | 35 | 33 | 57 | 51 | 57 |
| (%) | | | | | | | |
| p-value | 0.0021 | 0.0161 | 0.0065 | 0.0086 | 0.0352 | 0.0133 | 0.0074 |

Although the assays definitely detected different forms of circulating hOC, their performance in measuring the serum panel was almost identical. The IFMAs were even more effective in discriminating postmenopausal group from premenopausal group and also postmenopausal group under HRT from postmenopausal control group when measured in urine samples.

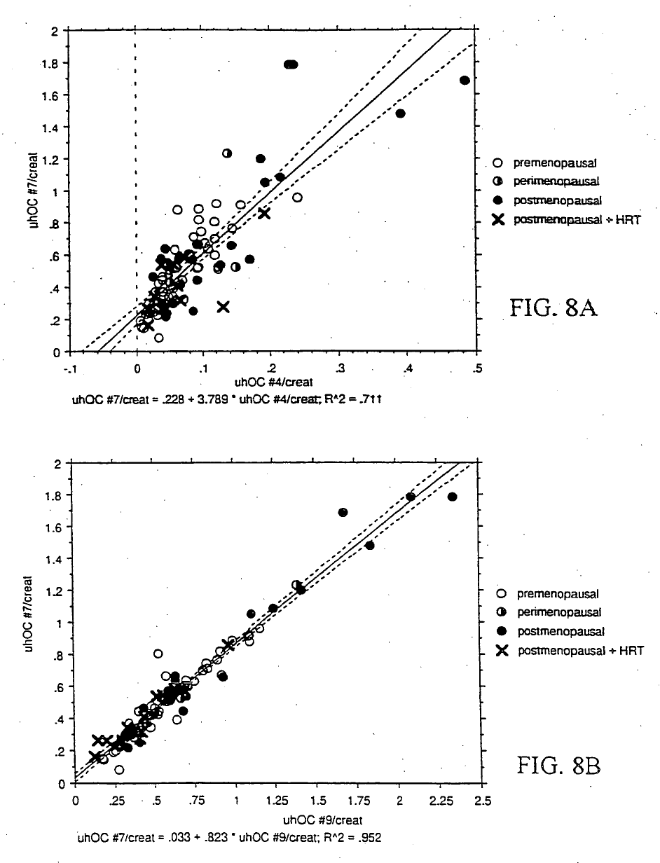

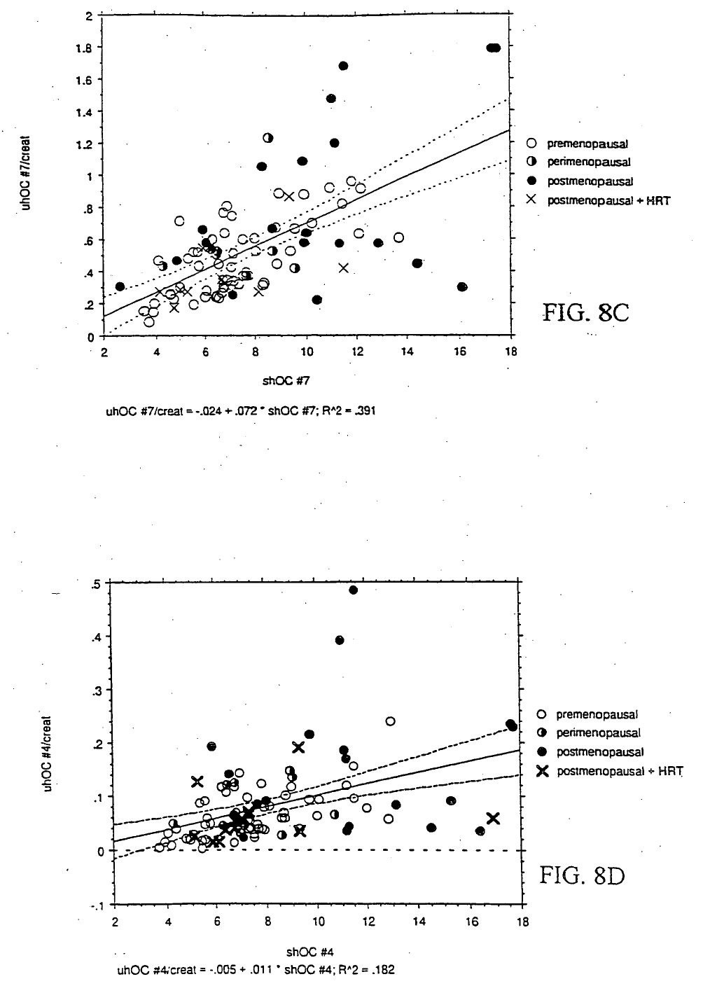

The serum hOC assays correlated well with each other. On the contrary, differences between urine assays were observed. As explained in section 5 the assay #4 recognizes a slightly different fragment in urine than assays #7 and #9. This might explain the weaker correlation between the assays #4 and #7 (r=0.843, FIG. 8A) than between assays #7 and #9 (r=0.976, FIG. 8B) as measured in the urine samples. When comparing the hOC values in urine and serum samples the correlation was remarkably lower although measured by the same Mab combination. Correlations between urine and serum samples in assay #7 (r=0.625) and assay #4 (r=0.427) are illustrated in FIGS. 8C and 8D, respectively. All the correlations were significant (p<0.001) and have been summarized in Table 5.

TABLE 5
Correlations between the hOC assays as measured in serum and
urine samples from adult females. All the correlations are significant (p < 0.001)

| | shOC #2 | shOC #4 | shOC #7 | shOC #9 | uhOC #4/creat | uhOC #7/creat | uhOC #9/creat |
|---|---|---|---|---|---|---|---|
| shOC #2 | 1.000 | .823 | .867 | .870 | .422 | .521 | .558 |
| shOC #4 | .823 | 1.000 | .932 | .929 | .427 | .555 | .572 |
| shOC #7 | .867 | .932 | 1.000 | .989 | .504 | .625 | .648 |
| shOC #9 | .870 | .929 | .989 | 1.000 | .519 | .653 | .671 |
| uhOC #4/creat | .422 | .427 | .504 | .519 | 1.000 | .843 | .824 |
| uhOC #7/creat | .521 | .555 | .625 | .653 | .843 | 1.000 | .976 |
| uhOC #9/creat | .558 | .572 | .648 | .671 | .824 | .976 | 1.000 |

It will be appreciated that the methods of the present invention can be incorporated in the form of a variety of embodiments, only a few of which are disclosed herein. It will be apparent for the specialist in the field that other embodiments exist and do not depart from the spirit of the invention. Thus, the described embodiments are illustrative and should not be construed as restrictive.

1. An isolated fragment of gamma-carboxylated osteocalcin, said gamma-carboxylated osteocalcin consists of the amino acid sequence Tyr-Leu-Tyr-Gln-Trp-Leu-Gly-Ala-Pro-Val-Pro-Tyr-Pro-Asp-Pro-Leu-Glu-Pro-Arg-Arg-Glu-Val-Cys-Glu-Leu-Asn-Pro-Asp-Cys-Asp-Glu-Leu-Ala-Asp-His-Ile-Gly-Phe-Gln-Glu-Ala-Tyr-Arg-Arg-Phe-Tyr-Gly-Pro-Val (SEQ ID NO:2), wherein at least one of the glutamic acids in position 17, 21 and 24 is gamma-carboxylated and said fragment is a peptide selected from the group consisting of
(i) a peptide consisting of amino acids 6-30 of said gamma-carboxylated osteocalcin; and
(ii) a peptide consisting of amino acids 7-30 of said gamma-carboxylated osteocalcin.

2. The fragment of claim 1, wherein all three of said glutamic acids are gamma-carboxylated.

3. The fragment of claim 1, wherein the peptide is (i).

4. The fragment of claim 1, wherein the peptide is (ii).

5. The fragment of claim 2, wherein the peptide is (i).

6. The fragment of claim 2, wherein the peptide is (ii).

7. A method for the measurement of the rate of bone turnover (formation and/or resorption) and/or for the investigation of metabolic bone disorders in an individual, comprising determining the quantity of gamma-carboxylated osteocalcin or gamma-carboxylated osteocalcin fragment in a urine sample,
wherein said gamma-carboxylated osteocalcin consists of the amino acid sequence Tyr-Leu-Tyr-Gln-Trp-Leu-Gly-Ala-Pro-Val-Pro-Tyr-Pro-Asp-Pro-Leu-Glu-Pro-Arg-Arg-Glu-Val-Cys-Glu-Leu-Asn-Pro-Asp-Cys-Asp-Glu-Leu-Ala-Asp-His-Ile-Gly-Phe-Gln-Glu-Ala-Tyr-Arg-Arg-Phe-Tyr-Gly-Pro-Val (SEQ ID NO:2), wherein at least one of the glutamic acids in position 17, 21 and 24 is gamma-carboxylated and
wherein said said gamma-carboxylated osteocalcin fragment is a peptide selected from the group consisting of (i) a peptide consisting of amino acids 6-30 of said gamma-carboxylated osteocalcin; and (ii) a peptide consisting of amino acids 7-30 of said gamma-carboxylated osteocalcin.

8. The method of claim 7, wherein the quantity of the gamma-carboxylated osteocalcin or the gamma-carboxylated osteocalcin fragment is determined by (a) contacting the urine sample with (i) a first monoclonal antibody or recombinant antibody fragment which bind to a first epitope on said gamma-carboxylated osteocalcin or gamma-carboxylated osteocalcin fragment and (ii) a second monoclonal antibody or recombinant antibody fragment which bind to a second epitope on said gamma-carboxylated osteocalcin or gamma-carboxylated osteocalcin fragment and (b) measuring the amount of bound antibodies or antibody fragments.

9. The method of claim 8, wherein all three of said glutamic acids are gamma-carboxylated.

10. The method of claim 8, wherein the quantity of gamma-carboxylated osteocalcin is determined.

11. The method of claim 8, wherein the quantity of gamma-carboxylated osteocalcin fragment is determined and the peptide is (i).

12. The method of claim 8, wherein the quantity of gamma-carboxylated osteocalcin fragment is determined and the peptide is (ii).

13. The method of claim 9, wherein quantity of gamma-carboxylated osteocalcin is determined.

14. The method of claim 9, wherein the quantity of gamma-carboxylated osteocalcin fragment is determined and the peptide is (i).

15. The method of claim 9, wherein the quantity of gamma-carboxylated osteocalcin fragment is determined and the peptide is (ii).


SEQUENCE LISTING (4 sequences)

SEQ ID NO: 1 - DNA, 160 bp, Homo sapiens
  CDS (1)..(147)
tacctgtatcaatggctgggagccccagtcccctacccggatcccctggagcccaggagggaggtgtgtgagctcaatccggactgtgacgagttggctgaccacatcggctttcaggaggcctatcggcgcttctacggcccggtctaactgcagatgc

SEQ ID NO: 2 - protein, 49 aa, Homo sapiens
  peptide (1)..(49): Glu at residues 17, 21 and 24 may be gamma-carboxy-Glu
YLYQWLGAPVPYPDPLEPRREVCELNPDCDELADHIGFQEAYRRFYGPV

SEQ ID NO: 3 - DNA, 45 bp, Homo sapiens
  CDS (1)..(33)
atcgaaggtcgtgggatccccgggaattcatcgtgactgactgac

SEQ ID NO: 4 - protein, 11 aa, Homo sapiens
IEGRGIPGNSS